(19) United States
(12) Patent Application Publication
(10) Pub. No.: US 2008/0132830 A1
(43) Pub. Date: Jun. 5, 2008

(54) Separable solid dosage form administration system

(75) Inventors: Brian Hague, Salt Lake City, UT (US); Lynn J. Maland, Salt Lake City, UT (US)

Correspondence Address: CEPHALON, INC., 41 MOORES ROAD, PO BOX 4011, FRAZER, PA, 19355, US

(73) Assignee: Cephalon, Inc., Frazer, PA (US)

(21) Appl. No.: 11/998,706

(22) Filed: Nov. 30, 2007

Related U.S. Application Data
(60) Provisional application No. 60/872,177, filed on Dec. 1, 2006.

(51) Int. Cl. A61M 5/178; A61P 25/30; A61K 31/465

(52) U.S. Cl. 604/36; 514/343

(57) ABSTRACT

The invention described herein provides a solid dosage form administration system comprising: an oral solid dosage form containing an active ingredient; and a hand-held applicator; wherein the hand-held applicator is removably coupled to the dosage form. The system includes a reversible coupling structure permitting repeated engagement and disengagement of the dosage form component from the hand-held applicator component of the system. In one embodiment, the applicator is composed of a soft durometer flexible polymeric material. The invention can be used to administer various medicaments, and is especially useful in treatment of nicotine addiction.

### FIELD OF THE INVENTION

The invention relates to the pharmaceutical field. In particular, the invention pertains to pharmaceutical and therapeutic drug delivery systems.

### BACKGROUND OF THE INVENTION

Solid lozenge-on-stick type dosage forms are well known. See U.S. Pat. Nos. 5,855,908, 5,288,498 and 6,165,495, for example, which describe medicated lollipop structures for delivering medicaments to patients. Also known are tobacco substitute devices for delivering nicotine to users, wherein the nicotine is administered in the form of a nicotine-containing candy-like matrix. See, for example, U.S. Pat. Nos. 5,132,114 and 5,824,334, which describe lozenge-on-stick type delivery systems that can be used in nicotine withdrawal therapy.

One problem associated with lozenge-on-stick type drug delivery systems is that while the user can selectively remove and re-insert the dosage form and stick into the oral cavity in accordance with his or her comfort or appearance preferences, the user of such systems is nevertheless forced to endure the presence of the stick throughout the administration or delivery of the active ingredient. Another problem associated with current lozenge-on-stick drug delivery systems employing hard plastic sticks is their safety and oral comfort.

There exists a need in the field of oral medication delivery systems for oral delivery systems that afford the user options for oral application and accommodate individual preferences of appearance and comfort for oral medication delivery routes. There is a further need for oral delivery systems, including nicotine delivery systems used in smoking cessation treatment, that afford the user the option to simulate habitual oral behaviors associated with addiction as part of the treatment.

### SUMMARY OF THE INVENTION

The invention provides an oral solid dosage form, e.g., lozenge, coupled to a hand-held applicator, e.g., stick, that, by virtue of its construction, facilitates reversible coupling of the dosage form to the hand-held applicator, as opposed to separation between the two components by undue exerted “force” or breaking of the device. The selective coupling and separation capabilities associated with the system of the invention provides several advantages: the invention affords a number of comfort and appearance options to the user; enhances the hygiene of the deposit and/or withdrawal of the dosage form from the oral cavity; removes the need for an assembly step in manufacture and packaging of the system as compared to fixed dosage form on stick type products. The invention further provides an oral medication delivery system, such as a nicotine delivery system, that can simulate habitual oral behaviors associated with addiction as part of nicotine withdrawal treatment as an option to the user.

The invention provides a solid dosage form administration system comprising: an oral solid dosage form containing an active ingredient; and a hand-held applicator; wherein the hand-held applicator is removably coupled to the dosage form. The system can further comprise a reversible coupling structure to permit separation and optionally re-coupling of the applicator to the dosage form.

The invention also provides a method of administering a pharmaceutically or therapeutically active ingredient to an individual recipient thereof comprising the steps of: providing a solid dosage form administration system wherein the solid dosage form contains an active ingredient and is removably coupled to a hand-held applicator; inserting the solid dosage form into the oral cavity of the recipient; and separating the applicator from the dosage form thereby withdrawing the applicator from the oral cavity and permitting the dosage form to remain within the oral cavity and release the active ingredient.

The invention further provides a solid dosage form administration system for treating nicotine addiction comprising an oral solid dosage form containing nicotine, nicotine derivative or complex as an active ingredient; and a hand-held applicator wherein the applicator is removably coupled to the dosage form. In one embodiment, the nicotine derivative is nicotine polacrilex.

The invention also provides a method of treating nicotine addiction in an individual in need of the treatment comprising providing to an individual a solid dosage form administration system comprising: an oral solid dosage form containing nicotine, nicotine derivative or complex as an active ingredient, and a hand-held applicator, wherein the applicator is removably coupled to the dosage form.

The invention provides a solid dosage form administration system comprising an oral solid dosage form containing an active ingredient; and a hand-held applicator removably coupled to the dosage form and having a generally elongated configuration and being composed of a flexible polymeric material.

The invention also provides a solid dosage form administration system comprising an oral solid dosage form containing an active ingredient and effervescing agent; and a hand-held applicator having a generally elongated configuration. In one embodiment, the dosage form further comprises a pH adjusting agent.

In a further aspect, the invention provides the combination of the solid dosage form administration system together with a packaging system, the dosage form administration system comprising:
a) a hand held applicator; and
b) a plurality of solid oral dosage forms;
   wherein the applicator and the dosage forms are structured for reversibly separable coupling to one another; and
   the packaging system comprising:
c) a tray comprising a trough structured to accommodate one or more hand-held applicators, and comprising a plurality of cavities structured to receive and accommodate individual dosage forms placed therein; and
d) a lid sealably engaged with said tray.

Other advantages associated with the invention and its various embodiments, will become apparent from the following disclosure.

### BRIEF DESCRIPTION OF THE DRAWINGS

The invention is further illustrated by the following figures, with numerical references which remain consistent throughout the figures—none of which are intended to necessarily impart limitations to the invention:

FIG. 1 is an angled side view of a solid dosage form administration system showing the dosage form component separated from the hand-held applicator component, according to one embodiment of the invention.

FIGS. 2A, 2B and 2C are side views of end portions of hand-held applicator having coupling structures of the circumscribing threaded type, according to one embodiment of the invention.

FIGS. 3A, 3B and 3C are side views of end portions of hand-held applicator having coupling structures of the surface texturing type, according to one embodiment of the invention.

FIGS. 4A, 4B and 4C are side views of end portions of hand-held applicator having coupling structures of the recessed type, according to one embodiment of the invention.

FIGS. 5A, 5B and 5C are side views of end portions of hand-held applicator having coupling structures of the elongated structure type, according to one embodiment of the invention.

FIG. 6 is a side view of an end portion of a hand-held applicator having an over-molded coupling structure, according to one embodiment of the invention.

FIG. 7 is an angled top view of an assembly showing separated components, including a packaging system and oral delivery system, according to one embodiment of the invention.

### DETAILED DESCRIPTION OF THE INVENTION

As used herein, the term “about” refers to a range of values from ±10% of a specified value, and functional equivalents thereof unless otherwise specifically precluded. For example, the phrase “about 50 mg” includes ±10% of 50, or from 45 mg to 55 mg.

Generally, the invention provides an oral delivery system that affords the user options for oral application and accommodate individual preferences for immediate or sustained delivery medications. The removably coupled solid dosage form administration system of the invention permits the user to: 1) deposit the medication into the oral cavity as a lozenge by itself in a hands-free manner using the hand-held applicator; 2) deposit and retain the medication in the oral cavity as a lollipop-type assembly; and/or 3) selectively employ both methods as deemed comfortable or appropriate to remove or reattach the hand-held applicator—without requiring handling of the dosage form component. To separate the dosage form from the applicator in situ, the user can retain the dosage form within the oral cavity by gently biting onto the dosage form and pulling or rotating the applicator. To re-couple, the user can similarly position the dosage form between the teeth and orient the cavity opening toward the outside of the mouth, and re-insert the applicator end into the cavity. The hands-free capability of deposit and withdrawal from the oral cavity enhances the hygiene of dosage form delivery for both the recipient and the administrator (if a different individual).

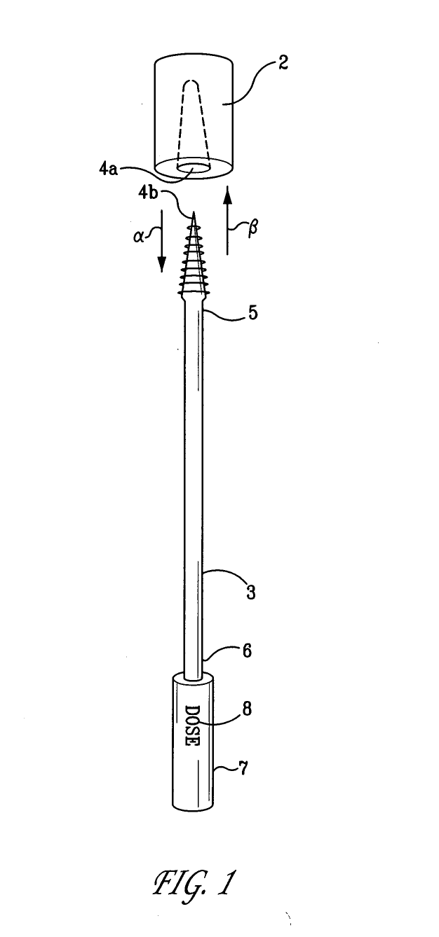

In one aspect, the invention includes a solid dosage form administration system comprising: an oral solid dosage form containing an active ingredient; and a hand-held applicator; wherein the hand-held applicator is removably coupled to the dosage form. Referring to FIG. 1, the solid dosage form 2 component is illustrated in separated condition from the hand-held applicator 3. The administration system of the invention affords the user the ability to engage the dosage form 2 onto the hand-held applicator 3 (movement shown as a), as well as disengage the two parts as well (movement shown as β. An important feature of the invention is that the dosage form 2 and hand-held applicator 3 are coupled via a reversible coupling structure (shown in FIG. 1 as a coordinating plug-and-socket structure 4A and 4B) that permits repeated engagement and disengagement throughout most of the delivery or usage event.

A variety of oral solid dosage forms can be used in accordance with the invention provided a portion or all of the dosage form has sufficient structural integrity to permit reversible coupling to the hand-held applicator component of the system. The dimensions, e.g., size and shape, of the dosage form can vary, provided the dimensions permit comfortable residence in the oral cavity of the recipient.

The dosage form administration system of the invention can be used to administer a wide variety of pharmaceutically or therapeutically active compounds and compositions. Examples of active ingredients that can be used with the invention include, but are not limited to, analgesics, anti-asthmatics, anti-inflammatory agents, antacids, anthelmintics, anti-arrhythmic agents, anti-bacterial agents, anti-coagulants, anti-depressants, anti-diabetics, anti-diarrheals, anti-epileptics, anti-fungals, anti-gout agents, anti-hypertensive agents, anti-malarials, anti-migrane agents, anti-muscarinic agents, anti-neoplastic agents, immunosuppressants, anti-parasitics, anti-protozoal agents, anti-rheumatics, anti-thyroid agents, anti-virals, anxiolytics, sedatives, hypnotics, neuroleptics, beta-blockers, cardiac inotropic agents, corticosteroids, cough suppressants, cytotoxics, decongestants, diuretics, enzymes, anti-Parkinsonian agents, gastrointestinal agents, histamine receptor antagonists, lipid regulating agents, local anaesthetics, neuromuscular agents, nitrates, anti-anginal agents, nutritional agents, opioid analgesics, oral vaccines, proteins, peptides and recombinants drugs, sex hormones, contraceptives, spermicides, stimulants, and the like.

The pharmaceutically active ingredient of the dosage form component can be formulated for a variety of administration routes. For example, the formulation can be prepared for oral transmucosal absorption of the active ingredient, and/or gastrointestinal absorption of the same.

In one embodiment, the system of the invention can comprise a dosage form comprising nicotine, a nicotine derivative or nicotine complex for treatment of nicotine addiction. For example, the dosage form can comprise nicotine polacrilex as the active ingredient. Suitable dosage forms for nicotine delivery include that described in Pinney et al., U.S. Pat. No. 6,893,654, the entire text of which is incorporated herein by reference, which describes a two-stage transmucosal oral delivery lozenge comprising a nicotine loading composition for immediate delivery and nicotine maintenance composition for prolonged delivery within the same lozenge.

The dosage form 2 component can be constructed as a uniform composition throughout. Alternatively, the dosage form component can be constructed to have a plurality of distinct regions having differing formulations, e.g., different dissolution rates or different dosage concentrations, for a given administration event. In another embodiment, the dosage form component can be constructed to administer combinations of different active ingredients, either simultaneously or in sequence corresponding to different regions or layers within the dosage form component. Multilayered or multi-region dosage forms can be prepared by molding or dipping techniques.

The dosage form component can be composed of any suitable formulation that can disintegrate or dissolve within the user's oral cavity when exposed to the user's saliva. Accordingly, the dosage form component can be formulated as a lozenge or hard-candy type dosage form containing a medicament. In an alternative embodiment, a drug-containing or drug coated non-dissolving dosage form substrate can be used in conjunction with the reversible coupling feature of the invention.

Alternatively, the dosage form can be formulated using ORAVESCENT® orally disintegrating dosage form technology (available from CIMA LABS™, Inc., Brooklyn Park, Minn.) and described in U.S. Pat. No. 6,200,604—the entire text of which is incorporated herein by reference. In general, this dosage form comprises an oral disintegrating solid dosage form that contains a medicament, effervescing agent in an amount sufficient to increase transmucosal absorption of the medicament, and can further comprise a pH adjusting substance. The term “effervescent agent” includes compounds which evolve gas, such as the chemical reaction upon initiated by exposure of the effervescent agent to water or saliva associated with the combination of a soluble acid source (citric acid) and carbon dioxide source (alkaline carbonate or bicarbonate).

When this dosage form is used in conjunction with the invention, the dosage form can be formed to receive and accommodate the hand-held applicator component of the invention. Thus, this embodiment affords the transmucosal absorption advantages associated with ORAVESCENT® technology in conjunction with the benefits associated with the removable hand-held applicator of the invention.

The hand-held applicator 3 component of the system of the invention can include a wide variety of configurations and structures provided manual management and manipulation of the system is permitted by the user. Although a wide variety of configurations are possible, the hand-held applicator 3 can have the general form of an elongated structure having a first end 5 and second end 6 (as shown in FIG. 1), e.g., rod, stick or stem similar to that typically found in a lollipop.

The hand-held applicator 3 can be constructed in accordance with conventional techniques and equipment readily available to those in the art. The applicator, for example, can be composed of a variety of materials provided the material affords the applicator sufficient structural rigidity for manual manipulation and coupling function. Such materials include, but are not limited to, plastic and paper. In a further embodiment, the applicator is composed of a disposable biodegradable material.

In a preferred embodiment of the invention, the hand-held applicator is composed of a relatively soft, semi-rigid, flexible or pliable polymeric material. Suitable flexible or pliable polymeric materials that can be used include those non-toxic polymers having a durometer value within the range of from about 40 to about 60 as measured using a Rockwell apparatus in accordance with ASTM D-2240. Flexible polymeric materials that can be used include, but are not limited to, polyvinyl acetate, polyvinylchloride, polystyrene, polypropylene, acetylbutyl styrene, and combinations thereof.

Several additional advantages of the invention are associated with the semi-rigid, flexible, pliable applicator embodiment. When the applicator is composed of a softer material, the risk of injury caused by lancing or puncture by the applicator is significantly reduced as compared to a rigid plastic material. Furthermore, the ability to break and fragment the applicator portion into smaller pieces, which could have presented a choking hazard, is significantly inhibited by use of a softer pliable material. Conversely, the softer, pliable material applicator affords the user the opportunity to safely bite or gnaw on the applicator as part of a smoking-associated behavior, which may aid some users in withdrawal success in a smoking cessation program.

Yet another advantage of the softer, pliable applicator is that the risk of cracking or fragmenting the dosage form component is reduced—both at the assembly stage and the delivery or usage stage. The softer material in this embodiment also enhances the safety of the system by reducing the likelihood of accidental puncture or lancing of the oral cavity that could be caused by the applicator component.

In a further embodiment, a portion of the hand-held applicator can comprise a grip, tab, or contain indicia or markings indicating dosage strength, brand names, and the like. Referring again to FIG. 1, the second end 6 of the hand-held applicator 3 comprises a grip 7 containing indicia 8 (represented as “DOSE”) thereon. In this regard, it is preferred that the second end 6 of the applicator 3 include a structure, such as a grip, handle or texturing, that also facilitates the separation or “pulling” of the applicator 3 to separate the applicator 3 from the dosage form 2.

The dosage form 2 component and hand-held applicator 3 component of the system of the invention are attached to one another through a reversible coupling structure (collectively illustrated in FIG. 1 as numerals 4A and 4B). One end (e.g. the first end 5) of the hand-held applicator 3 can further comprise a reversible coupling structure that interacts with a corresponding structure associated with the dosage form component that permits engagement and disengagement of the dosage form from the hand-held applicator.

The reversible coupling structure employed can take a variety of forms. The reversible coupling structures contemplated by this invention can take two general types—mechanically interfitting structures, friction-enhancing structures, and combinations thereof. A variety of reversible coupling structures are possible provided the structures permit or facilitate separation of the dosage form component from the hand-held applicator. Preferably, the re-insertion of the hand-held applicator into the dosage form component is also permitted. Suitable mechanically interfitting structures include, but are not limited to, threaded, snap-fit, rigid or semi-rigid annular rings, pegs, elongated ridges, and the like. Suitable friction-enhancing structures include, but are not limited to, combing, barbs, nodules, pebbling, roughening, texturing, pliable materials, and other like structures. It should be understood that the coupling structure can employ both mechanical and frictional interfitting attributes, and neither category is intended to be exclusive of the other.

In one embodiment, and as generally illustrated in the figures, the reversible coupling structure can comprise an interfitting or threaded pair of corresponding structures on each component such that separation of the applicator from the dosage form can be effectuated by simple outward rotation or twist of the components relative to one another along their shared longitudinal axis.

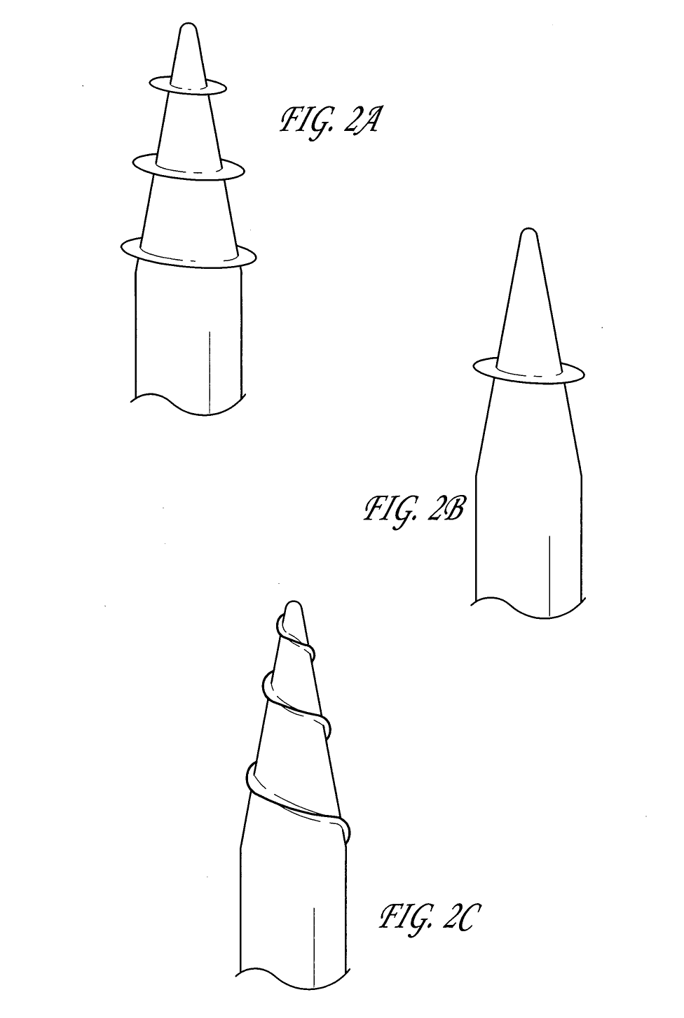

Referring to FIGS. 2A, 2B and 2C, there are illustrated hand-held applicator coupling structures of the circumscribing threaded type. FIG. 2A shows an end portion comprising a plurality of circumscribing annular protrusions, whereas FIG. 2B shows a single circumscribing protrusion or annular ring. FIG. 2C shows a spiral threaded structure.

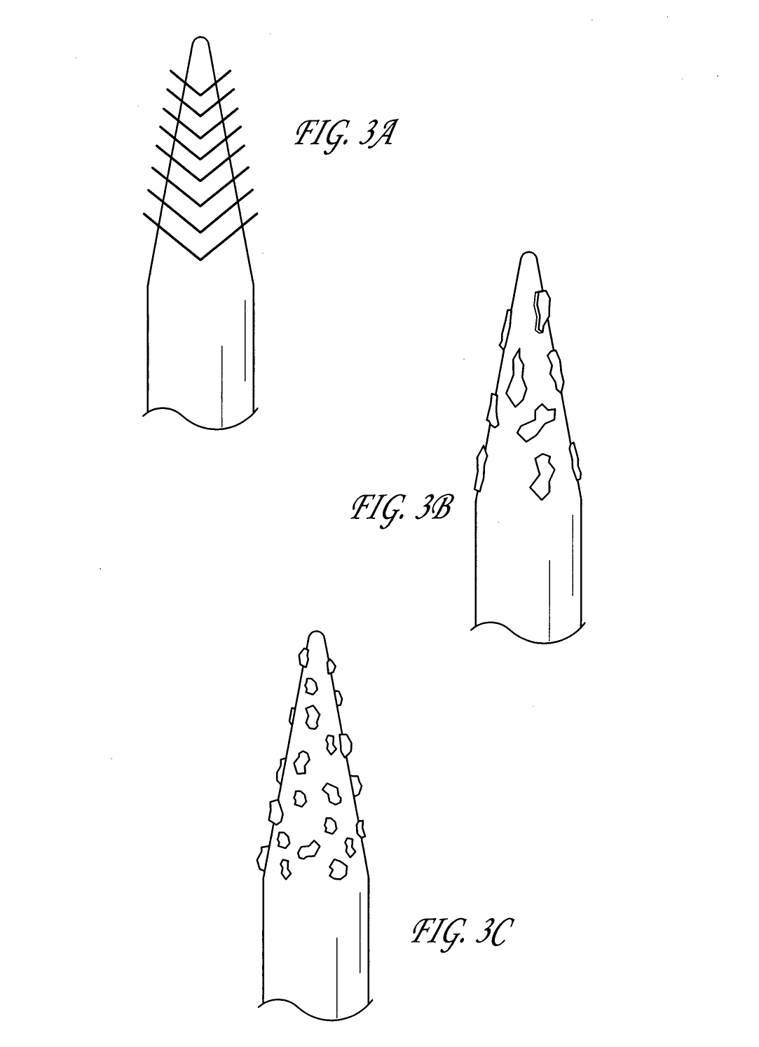

FIGS. 3A, 3B and 3C collectively illustrate various surface texturing embodiments of end portions of hand-held applicator components. FIG. 3A shows a combed exterior structure. FIGS. 3B and 3C show two types of surface texturing in the form of pebbling or nodular protrusions to enhance friction at the coupling interface.

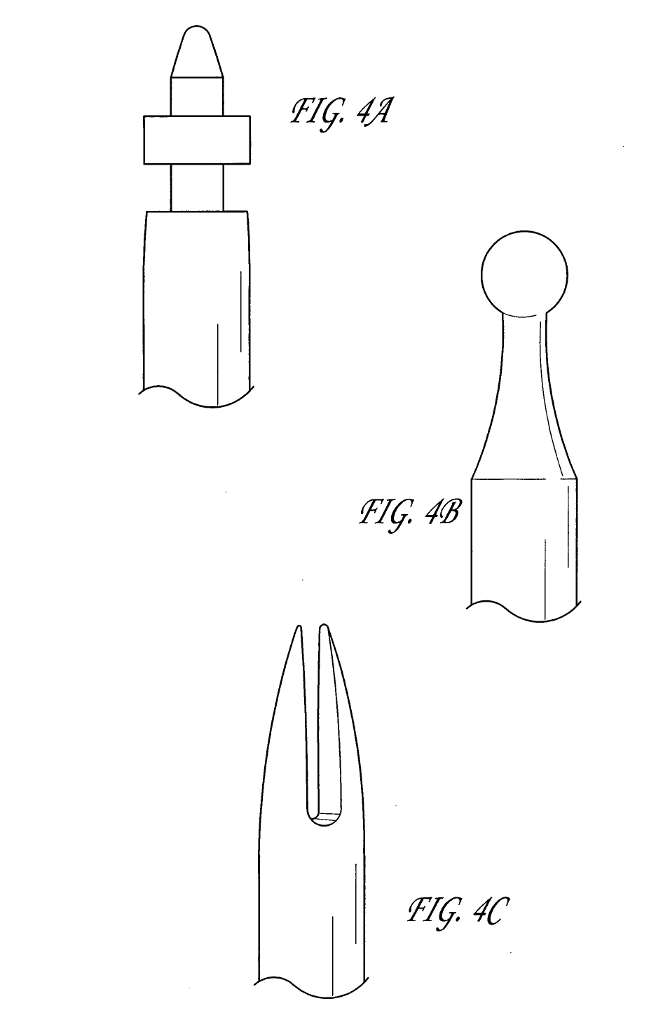

A protrusion structure can conversely be located within the cavity of the dosage form, and the corresponding coupling structure thereto located on the applicator. Referring now to FIGS. 4A and 4B, there are two illustrations of recessed-type structures in accordance with this embodiment. FIG. 4A shows annular recesses at the end portion, whereas FIG. 4B shows a necked prong structure. FIG. 4C shows a peg structure, which may interfit within the dosage form cavity and retain its position by tension or flexing of the peg by itself, or alternatively, the recess of the peg may interfit with a corresponding shelf (not shown) within the dosage form cavity.

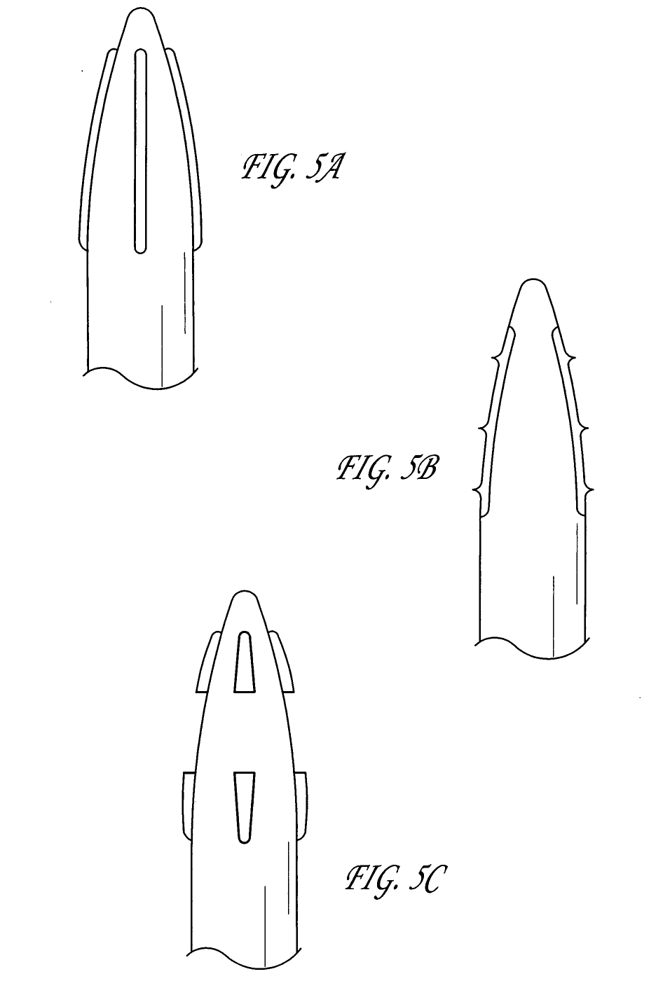

FIGS. 5A, 5B and 5C show several embodiments of elongated structures on the end portion of the applicator. FIG. 5A shows a plurality of longitudinal ridge structures substantially parallel to the longitudinal axis of the applicator body. Similarly, FIG. 5B shows an elongated structure further comprising barbs of protrusions on its surface, and FIG. 5C shows a plurality of “interrupted” elongated structures. According to the elongated structure embodiments, the hand-held applicator is simply slid in a longitudinal direction, in alignment with the longitudinal axis of the applicator, into the cavity of the dosage form component.

The reversible coupling structure is formed in part by preparing the dosage form component in contemplation of the corresponding hand-held applicator configuration. The dosage form itself can be prepared by conventional techniques, e.g., compression, molding and the like. For example, the dosage form component can be formed onto a mandrel or mold so as to create a cavity that functions as the receptacle for an end of the applicator component. (See, for example, FIGS. 1, 4A and 4B.) One preparation method involves mixing and blending the formulation ingredients in a suitable blender, and then compressing the resulting composition on a tablet press using tooling designed to create an aperture or cavity in the resultant dosage form. Using these methods, the interior dimensions of the resulting dosage form cavity accommodate the exterior dimensions and configuration of the hand-held applicator. In a further embodiment, the dosage form component can be molded or formed directly onto the coupling structure and applicator.

In another embodiment, the reversible coupling structure can be in the form of a soft, pliable interior core in a dosage form component having a harder surrounding region. Accordingly, the soft interior core can be in a form similar to a “gum”-like material, into which one end of the hand-held applicator can be reversibly inserted using relatively minor physical force.

In yet another embodiment, a coupling insert or inclusion can be positioned within or in association with the dosage form. Such insert can be formed from plastic, ceramic or other suitable material that functions as a structure to couple with the hand-held applicator. This embodiment is particularly useful in constructions wherein both the dosage form and the applicator have a harder, fracturable or brittle structural integrity, whereby the interface of the applicator and the dosage form is fortified through the insert or inclusion. Alternatively, this construct is useful when the dosage form has a soft structural integrity, and rigidity in the dosage form at the applicator interface of the reversible coupling structure can maintain the coupling structure—irrespective of the soft integrity of the dosage form.

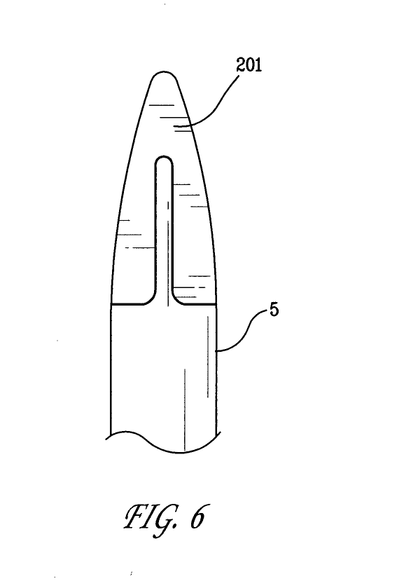

The hand-held applicator can be constructed of two or more different materials having different physical and/or chemical properties. In one embodiment, the end portion of the hand-held applicator component that interacts with the dosage form component can comprise a soft, elastomeric polymeric material. For example, a soft, elastomeric polymer 201 can be over-molded or otherwise coupled onto the hand-held applicator end as shown in FIG. 6. According to this embodiment, the softer material of the polymer 201 is inserted into a cavity formed in the dosage form component (not shown) thereby pliably fitting and accommodating the interior dimensions of the cavity. This embodiment is particularly advantageous when used in combination with brittle or friable dosage form compositions.

The hand-held applicator (and the corresponding coupling structure element associated with the applicator) can be manufactured using suitable natural or synthetic fiber, resin, rubber, metal, and polymeric materials, and the like. The applicator component can be manufactured by extrusion, injection molding, milling, casting techniques, and the like, into a rod-like or stick-like configuration that includes the desired coupling structure. The coupling structure associated with the applicator can also be formed directly from the applicator material or over-molded onto the applicator.

The applicator itself can comprise a secondary composition as an infusion or coating onto the applicator. For example, the secondary composition can comprise an additional pharmaceutical compound, breath freshener, and the like. According to this embodiment, when the dosage form has disintegrated or otherwise been consumed or dissolved, the secondary composition can be exposed and administered in supplement to the primary dosage form.

In another aspect, the invention involves a method of administering a pharmaceutically or therapeutically active ingredient to a recipient comprising: providing to the recipient a solid dosage form administration system comprising: an oral solid dosage form containing an active ingredient; and a hand-held applicator; wherein the hand-held applicator is removably coupled to the dosage form.

In one embodiment, the invention includes a solid oral dosage form administration system and corresponding method of treatment of nicotine addiction comprising administering to an individual in need of such treatment a solid dosage form administration system of the invention. The method comprises providing the system of the invention comprising an oral solid dosage form containing nicotine, nicotine derivative or nicotine complex as an active ingredient; and a hand-held applicator; wherein the hand-held applicator is removably coupled to the dosage form. Nicotine polacrilex can be used as the active ingredient in this treatment method.

Wherein the invention is used to treat nicotine addiction, the dosage form administration system of the invention affords the benefit of simulating the physical or behavioral aspect of the addiction, i.e., the oral placement and withdrawal of a cigarette. Accordingly, the oral dosage form component of the system can be formulated to mimic the typical residency of a cigarette and tobacco usage nicotine delivery parameters. For example, a tobacco-related nicotine delivery time of about 3 minutes to about 20 minutes can be mimicked by formulating the oral dosage form so as to provide nicotine (substitute) delivery over a about 3 minute to about 20 minute time period by controlling the dissolving rate of the dosage form.

Additionally, the hand-held applicator alone, dosage form alone, or the combination of both, can be dimensioned to mimic the dimensions, and/or appearance of, a (tobacco) cigarette. Thus, the dosage form administration system can have an overall cylindrical shape with a length of approximately 11 centimeters and a cross-sectional diameter of approximately 4 to 6 millimeters. Alternatively, the dosage form component itself can have a cylindrical shape having a exterior length of about 1 to about 3 centimeters, and a cross-sectional diameter of about 5 mm to about 12 mm. Irrespective of the dosage form component, the hand-held applicator component can vary and have a length of about 3 centimeters to about 9 centimeters. The cross-sectional diameter, or dimensions, of the entire hand-held applicator component or only a portion of the applicator, can be configured to be similar to a (tobacco) cigarette, e.g. 8 mm.

Whether the dosage form administration system of the invention is used to deliver a pharmaceutically active ingredient or nicotine as part of a smoking cessation product, the appearance of the dosage form component, hand-held applicator, or the combination of both, can be vary and be modified according to manufacturing or consumer preferences, for example. In addition to possible variations in the dimensions and shape of the system, flavoring and coloration of the individual components or assembly can be modified as well using conventional additives, materials and techniques readily available to those skilled in the art.

The dosage form administration system of the invention can be manufactured using conventional techniques and equipment readily available to those skilled in the pharmaceutical manufacturing field. When the dosage form is a medicated lozenge, for example, a variety of methods for preparing the dosage form component can be used, including dry-powder blending, wet granulation, co-melts or solid dispersions. The dosage form can be in the form of an oral transmucosal solid dosage form prepared using compressed powder, such as those described in Stanley et al. U.S. Pat. Nos. 4,863,737, 5,132,114, the full text of which are incorporated herein by reference.

Selection of the suitable or appropriate manufacturing technique will vary according to the particular dosage form ingredients and properties. For example, the ingredients and their associated individual or combined characteristics such as solubility, bulk density, pH, and the like, are determined. These ingredients, along with the active pharmaceutical ingredient, can be combined by blending directly or by high shear granulation and fluid bed drying. The combined mixture can then be compressed, molded, lyophilized in situ, or otherwise formed into the desired overall configuration, e.g., lozenge with aperture into or onto which the hand-held applicator can be affixed.

For manufacturing a sugar-containing hard candy oral transmucosal dosage form, the following process steps can be used:
a) dissolve solid sucrose in liquid dextrose within a heated vessel;
b) add the heat-stable drug in solid or solution and disperse within the sucrose/dextrose mixture;
c) stir the mixture and heat to a temperature of about 150° C.;
d) apply a vacuum to the mixture until the hard crack stage is reached;
e) add additional ingredients if desired, e.g., buffers, flavors, coloring agents, and the like;
f) dispense and mold into dosage units of predetermined shape and size using tooling that creates the reversible coupling structure to be located on the dosage form component;
g) cool and package alongside the hand-held applicator component.

Additional or secondary ingredients can be added to the dosage form component formulation, provided such additives are suitable for ingestion. Examples of such secondary ingredients that can be used include, but are not limited to, FD&C dyes and natural coloring agents, natural or synthetic flavoring agents, artificial and natural sweeteners, and the like.

### Packaging System

The dosage form administration system of the invention can be packaged individually or as a plurality. Furthermore, the system can be presented in the form of a series of sequential dosage amounts to be administered over a time period, e.g., several days. Thus, the amount of pharmaceutically active ingredient within the dosage forms can be constant for multiple delivery episodes, or tapered increasingly or decreasingly over a total delivery time frame. In this embodiment, the user can select the desired dosage form, e.g., lozenge, from a series within a package using the hand-held applicator and couple the applicator to the chosen dosage form unit for the given delivery episode.

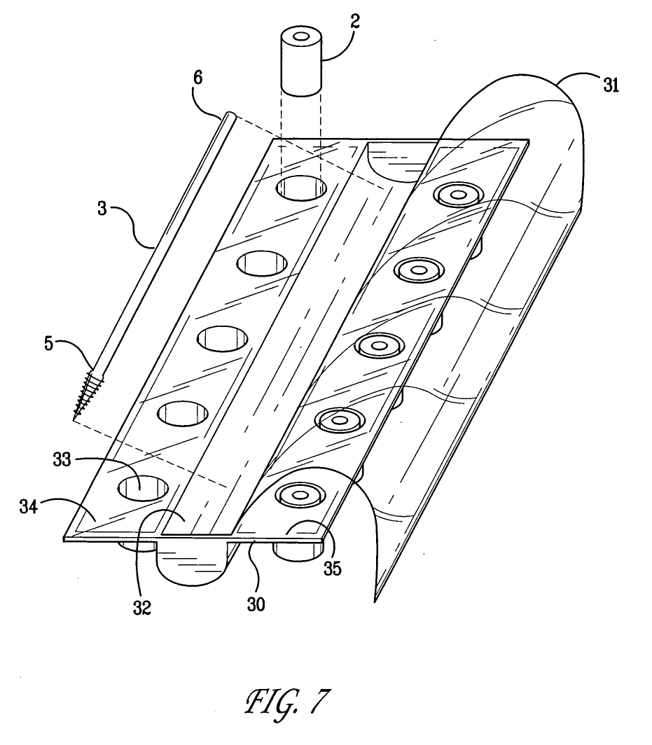

In a further aspect, the invention provides the combination of the solid dosage form administration system together with a packaging system, the packaging system comprising:
a) a tray comprising a trough structured to accommodate one or more hand-held applicators, and comprising a plurality of cavities structured to receive and accommodate individual dosage forms placed therein; and
b) a lid sealably engaged with the tray.
   One example of a packaging system that can be used with the invention is illustrated in FIG. 7. Referring now to FIG. 7, a thermoformed plastic tray 30 is shown containing a partially removed peelable lid 31 as a lid sealably engaged with the tray, along with a solid dosage form administration system (hand-held applicator 3 and solid dosage form 2). The tray 30 can have a generally planar configuration that includes a trough 32 positioned along the central longitudinal axis (not shown) of the tray 30. The trough 32 can be structured to accommodate one or more hand-held applicators 3. Thus, the dimensions of the trough 32, e.g., length, width, depth, can be selected to accommodate the dimensions of the hand-held applicator 3 and their number desired for residence within the trough 32.

Alongside both sides of the trough 32 are shown a plurality of cavities 33 structured to receive and accommodate individual dosage forms 2 placed therein. As illustrated, the cavities 33 are shown located on two planar flanges 34 on opposing sides of the trough 32. The interior dimensions of the cavity(ies) 33, e.g., height or length, width or diameter, are selected to accommodate the outer dimensions, e.g., length and width or diameter, or configuration, of the dosage form(s) to be retained therein.

Suitable tray materials include metals and metallic alloys, such as aluminum, as well as thermoformable or thermoplastic polymers, including but not limited to polyvinylchloride (PVC), polyethylene (HDPE or LDPE), and the like. The tray material can be opaque or transparent, colored, textured, and such. Due to the nature of the dosage forms to be contained, tray materials that inhibit or prevent the ingress of environmental humidity or moisture are preferred. The tray component can be manufactured using conventional die or molding techniques readily available to those skilled in the pharmaceutical and medical device packaging arts.

The lid sealably engaged with the tray can take a variety of forms and structures, provided access of ingress of the external environment is at least inhibited by its structural cooperation with the tray component. This is important because the dosage form component can be of such a nature as to be adversely affected by external humidity and moisture. Thus, sealable engagement is an important aspect of the packaging system. The lid component to be sealably engaged with the tray can be constructed in a variety of forms and from a variety of materials. For example, the lid can be hingedly attached to the tray as a flap or cover, can be a longitudinal slidable cover or encasement within which the tray can be housed, or can be a peelable lid (as shown). Of course, the tray and lid structure are selected to structurally cooperate and engage one another.

When a peelable lid 31 is used, the material can be composed of paper or other flexible fibrous material, (e.g., TYVEK®), plastic film-laminated material, flexible thin plastic, metal or aluminum foil, and the like. Lidding materials can further be opaque or transparent. Referring now to FIG. 7, a peelable lid 31 can be peelably adhered to the circumscribing perimeter region 35 of the tray 30. The peelable lid 31 can be attached to the perimeter 35 using a variety of techniques and materials readily available to those in the medical packaging art, including ultrasonic welding, hot melt adhesives, and the like.

In an alternative embodiment, the packaging system can comprise a plurality of individual lids associated with one or more cavities on the tray. According to this embodiment, the user can avoid opening the remaining cavities and exposing the dosage forms therein when desiring to withdraw only one dosage form at a time.

In use, the user detaches or otherwise opens the lid to expose the hand-held applicator and at least one dosage form. The user can then couple the applicator to the dosage form and remove the dosage form from the cavity without touching the dosage form. Should the user decide that an independent dosage form or lozenge is preferable, the user can simply detach the applicator and return the applicator to the trough of the packaging system—without the need to handle or touch the dosage form at any point. Overall, the packaging system of the invention can provide yet another benefit as to mimicking behavioral aspect of addiction by affording the user an object withdrawal routine in place of the routine of withdrawing a cigarette from a cigarette pack.

### EXAMPLE

### Example 1

### Preparation of Dosage Form and Removable Hand-Held Applicator System

The manufacture of a dosage form administration system of the invention generally comprises three major steps: 1) Preparation of the formulation; 2) Formation of the dosage form; and 3) Assembly of the dosage form and applicator.

a) Preparation of Formulation

A mannitol-based placebo formulation was prepared by initially preparing a blend. Granular mannitol was weighed into two parts (1 kg and remaining large portion), and each of the remaining ingredients were weighed and placed in suitable containers. Approximately half of the large portion of granular mannitol was screened through a 14 mesh screen while adding the screened mannitol into a 50 L stainless steel mixing bin. The rest of the raw ingredients were screened through a 14 mesh screen and likewise added into the bin (with the exception of 1 kg of granular mannitol and magnesium stearate). The rest of the granular mannitol was then screened and added on top. The composition was mixed using the mixer set at a rate of 9 rpm for a period of 60 minutes.

The magnesium stearate and 1 kg granular mannitol were then co-screened through a 20 mesh screen and added into the blend. The blend was then mixed for an additional 5 minutes at 9 rpm.

The resulting blend had the formulation set forth in the following table.

TABLE 1
Mannitol Based Placebo Formulation (20 kg)

| | Amount | Amount | Amount |
|---|---|---|---|
| Ingredient: | (mg)/Unit | (w/w %) | (kg)/20 kg |
| Granular mannitol Mannogem ™ | 958.7 | 47.90 | 9.59 |
| 2080 | | | |
| Mannogem ™ EZ (spray-dried | 410.9 | 20.50 | 4.11 |
| mannitol) | | | |
| Polyplasdone ™ XL (N-vinyl- | 150.0 | 7.50 | 1.50 |
| 2-pyrrolidone homopolymer) | | | |
| Avicel ™ 101 (microcrystalline | 400.0 | 20.0 | 4.00 |
| cellulose) | | | |
| Sucralose | 5.0 | 0.25 | 0.050 |
| Acesulfame K | 5.0 | 0.25 | 0.050 |
| Bubble gum, flavor PWD #730 | 30.0 | 1.50 | 0.300 |
| Prosweet ™ N&A flavor PWD | 20.0 | 1.00 | 0.200 |
| D&C Red #30 Aluminum Lake | 0.4 | 0.02 | 0.004 |
| Magnesium stearate | 20.0 | 1.00 | 0.200 |
| Total: | 2000.0 | 100.00 | 20.00 |

MANNOGEM ™ 2080 and MANNOGEM ™ EZ available from SPI Pharma, New Castle, Delaware; POLYPLASDONE ™ XL available from International Specialty Products; AVICEL ™ 101 available from FMC Biopolymer, Philadelphia, Pennsylvania; Acesulfame Potassium C₄H₄KNO₄S available from Chempoint, Bellevue, Washington; Bubble gum flavor and PROSWEET ™ N&A available from Virginia Dare, Brooklyn, New York.

b) Formation of the Dosage Form

A placebo dry solid dosage form was prepared using the above formulation by compression technique. Compression was performed using a R&D rotary Fette press. The press was set up for sample batch using 10 punches (and 19 blanks) and using a double stage feeder. The blend prepared above was compressed into tablets with a target weight set for 2 grams, and a length of approximately 0.685 inches. The resulting tablet was formed with a cavity dimensioned to accommodate a portion of the applicator.

Preferably, a combination of granular mannitol and spray dried mannitol in a ratio of 7:3 can be used to achieve good flow, good compressibility with little or no capping or breaking, short disintegration time (less than 2 minutes), and friability of less than 1%.

During manufacture, the dosage form can be formed in conjunction with apparatus and tooling for forming an internal cavity in the dosage form component for receiving the applicator within. The dimensions, i.e., length, width, circumference, tapering, etc. can be modified depending on the dimensions of the applicator end and the nature of the dosage form and its dimensions. To facilitate receipt and removal of the applicator, however, it is preferred that the tooling be configured to create a tapered, generally conical cavity within the central region of the dosage form (as shown in FIG. 1, for example).

c) Assembly of Dosage Form and Applicator

The dosage form component and applicator component can be initially coupled at the time of manufacture and packaging. Alternatively, the dosage form and applicator can be presented to the user or packaged as separate components for coupling at the time of use. Once coupled, the two components can be separated and re-coupled, either by grasping each component by hand, or in situ, by retaining the dosage form component between the user's teeth with the cavity oriented outward for receipt of the applicator end. The hand-held applicator can be composed of flexible polypropylene having a durometer value between about 40 and 60 as measured according to ASTM D-2240.

### INDUSTRIAL APPLICABILITY

The solid dosage form administration system can be used to delivery a variety of pharmaceutical and veterinary medicaments. The invention provides usage options for the user by permitting the presence or absence of the applicator, as well as manufacturing and safety advantages by virtue of its structure.

The invention has been described herein above with reference to various and specific embodiments and techniques. It will be understood, however, that reasonable variations and modifications can be made from such embodiments and techniques without significant departure from either the spirit or scope of the invention defined by the following claims.

What is claimed is:

1. A solid dosage form administration system comprising:
a) an oral solid dosage form containing an active ingredient; and
b) a hand-held applicator;
wherein said hand-held applicator is removably coupled to said dosage form.

2. The system according to claim 1, wherein said applicator has a generally elongated configuration having a first end and second end, wherein said second end comprises a reversible coupling structure to reversibly couple said applicator to said dosage form.

3. The system according to claim 1, wherein said hand-held applicator is composed of a flexible polymeric material.

4. The system according to claim 5, wherein said flexible polymeric material has a durometer value of from about 40 to about 60.

5. A method of administering a pharmaceutically or therapeutically active ingredient to an individual recipient thereof, said method comprising the steps of:
a) providing a solid dosage form administration system wherein said solid dosage form contains an active ingredient and is removably coupled to a hand-held applicator;
b) inserting said solid dosage form into the oral cavity of said recipient; and
c) separating said hand-held applicator from said dosage form thereby withdrawing said applicator from said oral cavity and permitting said dosage form to remain within said oral cavity of said recipient thereby releasing said active ingredient.

6. The method according to claim 5, wherein said applicator is composed of a flexible polymeric material.

7. The method according to claim 6, wherein said flexible polymeric material has a durometer value from about 40 to about 60.

8. A solid dosage form administration system for treating nicotine addiction comprising:
a) an oral solid dosage form containing nicotine, nicotine derivative or nicotine complex as an active ingredient; and
b) a hand-held applicator;
wherein said hand-held applicator is removably coupled to said dosage form.

9. The system according to claim 8, wherein said applicator is composed of a flexible polymeric material.

10. The system according to claim 9, wherein said flexible polymeric material has a durometer value from about 40 to about 60.

11. A method of treating nicotine addiction to an individual in need of said treatment comprising providing to said individual a solid dosage form administration system comprising:
a) an oral solid dosage form containing nicotine, nicotine derivative or nicotine complex as an active ingredient; and
b) a hand-held applicator;
wherein said hand-held applicator is removably coupled to said dosage form.

12. The method according to claim 11, wherein said applicator is composed of a flexible polymeric material.

13. The method according to claim 12, wherein said flexible polymeric material has a durometer value from about 40 to about 60.

14. A combination of solid dosage form administration system together with a packaging system, said dosage form administration system comprising:
a) a hand held applicator; and
b) plurality of solid oral dosage form;
wherein said applicator and said dosage form are structured for reversibly separable coupling to one another; and
said packaging system comprising:
c) a tray comprising a trough structured to accommodate one or more hand-held applicators, and comprising a plurality of cavities structured to receive and accommodate individual dosage forms placed therein; and
d) a lid sealably engaged with said tray.
